(19) United States
(12) Patent Application Publication
(10) Pub. No.: US 2005/0095246 A1
(43) Pub. Date: May 5, 2005

(54) Techniques to treat neurological disorders by attenuating the production of pro-inflammatory mediators

(75) Inventor: Lisa L. Shafer, Stillwater, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/972,157

(22) Filed: Oct. 22, 2004

Related U.S. Application Data
(60) Provisional application No. 60/514,137, filed on Oct. 24, 2003.

(51) Int. Cl. A61K 39/395; A61K 9/22

(52) U.S. Cl. 424/145.1; 604/890.1

(57) ABSTRACT

Methods and devices to attenuate tumor necrosis factor (TNF) and other pro-inflammatory mediators in the CNS to treat neurological, neurodegenerative, neuropsychiatric disorders, pain and brain injury are described. More particularly, TNF blocking agents that target intracellular signals and downstream effects associated with the production and secretion of TNF are described. Devices described include therapy delivery devices comprising a reservoir capable of housing a TNF blocking agent and a catheter operably coupled to the device and adapted to deliver the TNF blocking agent to a target site within a subject.

### RELATED APPLICATIONS

This application claims the benefit of priority to U.S. Provisional Application Ser. No. 60/514,137, filed Oct. 24, 2003, which application is incorporated herein by reference in its entirety.

### FIELD

This invention relates to medical devices and methods for attenuating pro-inflammatory mediators, particularly for treatment of neurological, neurodegenerative, neuropsychiatric disorders, pain and brain injury.

### BACKGROUND

Neurodegeneration that is characteristic of neurodegenerative disease and traumatic brain injury may progress even when the initial cause of neuronal degeneration or insult has disappeared. It is believed that toxic substances released by the neurons or glial cells may be involved in the propagation and perpetuation of neuronal degeneration. Neuronal degeneration and other disease pathology in the brain has been attributed to the toxic properties of proinflammatory cytokines, such as tumor necrosis factor alpha or beta (TNF), interleukin (IL)-1 beta, and interferon (IFN)-gamma. Therapies aimed at inhibiting proinflammatory cytokines, particularly TNFα, may attenuate the pathology associated with chronic pain, neurodegenerative diseases, traumatic brain injury and abnormal glial physiology. Furthermore, inhibiting the constitutive levels of pro-inflammatory cytokines may provide a prophylactic therapy for individuals at risk for, or at early stages of, a certain disease or condition of the brain.

Several TNF blocking agents have been developed for systemic administration and are approved for treating various diseases of the periphery such as rheumatoid arthritis and Crohn's disease. Currently available blocking agents act on soluble, extracellular TNF or TNF receptors. These agents are administered in the periphery and are not capable of penetrating the blood-brain-barrier. While these agents are effective for the above-mentioned indications, this class of TNF blocking agents is associated with the risk of serious side-effects, such as opportunistic infections, immuno-supression and demyelinating diseases. Moreover, recent reports have led to the counter-indication of systemic, chronic use of some of the commercially available TNF blocking agents in individuals with a history of central nervous system disorders.

Despite this counter-indication, the use of such TNF blocking agents to treat neurological and neuropsychiatric disorders has recently been suggested. US 2003/0049256A1 and WO 03/271 8A2 (Tobinick) discuss the administration of cytokine antagonists via intranasal and perispinal routes of administration as a way of treating neurological or neuropsychiatric disorders or diseases. The Tobinick patents do not disclose the administration of agents to block the intracellular signal transduction cascade involved in the production and cellular secretion of TNF and other cytokines. They also do not disclose administration of a combination of extracellualar antagonists, cell-surface receptor antagonists, with agents targeting the intracellular signal transduction cascade. They do not disclose the administration of such agents complexed with a depot. Furthermore, methods or devices for the targeted administration of such agents intraventricularly or to the intraparenchymal brain tissue have not been described.

The agents described by Tobinick are limited to blocking extracellular TNF and its extracellular or cell surface receptors. The TNF blocking agents discussed by Tobinick form complexes composed of soluble TNF and its blocking agent. In the periphery, these complexes are broken down and eliminated via phagocytic clearance. This mechanism of action is efficacious and therapeutic in several peripheral diseases. However, the brain does not have these same clearance mechanisms. Therefore, it is possible that there is a greater potential for the toxic TNF molecule to be stabilized by the blocking agents, leading to greater toxic effects in the brain tissue. The method disclosed by Tobinick is depicted by #1 in the schematic of TNF signal transduction presented in FIG. 1.

Furthermore, in the periphery, some currently available blocking agents ultimately engage the TNF receptor and initiate apoptosis, or programmed cell death, in the TNF producing cell. This is a desired effect of a TNF blocking therapy in the periphery because death of activated cells is beneficial and because these cells are capable of replenishing themselves. However, when these same agents are applied to cells of the central nervous system (CNS) and their mechanism of action results in apoptosis of neurons, a deleterious effect can occur. Because neurons are substantially incapable of regenerating themselves, apoptosis of neurons is detrimental to the brain.

Moreover, since several different brain cell types produce TNF and express TNF receptors, the indiscriminant blocking of TNF receptors on a cell surface may result in non-target cell tissue binding. This non-specific effect may have serious consequences in the brain. Compared to the periphery, brain tissue is less “immunocompetent” and as a result, this non-specific effect cannot be compensated for and may result in exacerbated conditions.

TNFα is a non-glycosylated polypeptide that exists as either a transmembrane or soluble protein. TNFα increases production of pro-inflammatory molecules and several adhesion molecules resulting in the initiation of an inflammatory cascade. Frequently, the TNF-initiated cascade has deleterious effects at the cellular, tissue and organ level. Inhibition of TNF synthesis can be achieved by several means including: (1) inhibition of transcription; (2) decrease of the mRNA half-life; (3) inhibition of translation, and (4) inhibition of signaling molecules both before and after the transcription of the TNF gene product.

The TNFα signal is initiated by binding to the TNF receptors on a cell's surface. There are two TNFα receptors (TNFRI and TNFRII). Several signal transduction events occur following the dimerization of the two receptors. The two best-characterized TNF-induced effects are apopotosis and NFKB activation. Apoptosis results in cell death. NFkB activation, through a serious of additional events results in the production of a variety of other effector molecules that further propagate an inflammatory cascade (ie Il-1, HMGB-1, more TNF, etc). These effects are referred to as “downstream effects” of the TNF initiated cascade.

The pathway of downstream effects initiated by TNF can be regulated at several points by administering a variety of biologic or small molecule therapeutic agents either alone or in combination with each other. Many of these agents have been developed or are currently in development for peripheral administration to treat peripheral diseases and conditions that are manifested by elevated TNF. However, the administration of these types of agents to targeted areas in the brain or spinal cord has not been suggested previously as a way to treat or prevent conditions associated with brain injury, pain, neurological, neuropsychiatric, and neurodegenerative disease.

TNF and TNF receptors are expressed in the brain by astrocytes, neurons, monocytes, microglia and blood vessels. Biologic or small molecule drug therapeutic agents targeting the intracellular TNF cascade in these cell populations may have a therapeutic or prophylactic effect in diseases and conditions of the central nervous system.

The production, release, and subsequent action of TNF depends on an extensive intracellular signal transduction cascade. The administration of intracellular TNF signal transduction modulating agents to the brain for the therapeutic and prophylactic benefit has not previously been described. Additionally, the administration of a combination of intracellular and extracellular TNF modulating agents to the brain for therapeutic and prophylactic benefit has not previously been described.

### BRIEF SUMMARY

This disclosure describes targeting intracellular signals and downstream effects associated with the production and secretion of TNF and describes methods and devices to attenuate tumor necrosis factor (TNF) and other pro-inflammatory mediators in the CNS to treat neurological, neurodegenerative, neuropsychiatric disorders, pain and brain injury. Potentially safer and more efficacious means of administration, as well as potentially safer and more efficacious agents aimed at blocking TNF, its signal transduction cascade, and its downstream mediators are discussed. Some of these agents are being considered as second generation therapies to the current, commercially available extracellular TNF blocking agents for use in peripheral diseases. However, these agents have not been described for use in the brain or spinal cord or to treat CNS disorders.

An embodiment of the invention provides a system for treating a CNS disorder associated with a proinflammatory agent in a subject in need thereof. The system comprises a device having a reservoir adapted to house a therapeutic composition, a catheter coupled to the device and adapted for administering the therapeutic composition to the CNS of the subject, and a CNS disorder treating amount of a therapeutic composition. The system may also include a sensor. The senor may be coupled to a device to adjust one or more infusion parameters, for example flow rate and chronicity. The sensor may be capable of detecting a dysfunctional immune or sickness response, or whether an immune response has been attenuated or enhanced, and the like. The therapeutic composition comprises an intracellular TNF modifying agent in an amount effective to treat the CNS disorder. The therapeutic agent may be administered directly to the CNS (intrathecally, intracerebroventricularly, intraparenchymally, etc.) or may be administered peripherally, such as perispinally or intranasally.

In embodiments, the invention provides systems and methods for the administration of a therapeutic composition comprising a combination of extracellular and intracellular TNF modifying agents. In an embodiment, a system for administration of a therapeutic composition comprising a combination of extracellular and intracellular TNF modifying agents is a “controlled administration system”. A “controlled administration system” is a direct and local administration system to deliver the combination of agents. A controlled administration system may be a depot or a pump system, such as an osmotic pump or an infusion pump. An infusion pump may be implantable and may be a programmable pump, a fixed rate pump, and the like. A catheter is operably connected to the pump and configured to deliver the combination of agents to a target tissue region of a subject. A controlled administration system may be a pharmaceutical depot (a pharmaceutical delivery composition) such as a capsule, a microsphere, a particle, a gel, a coating, a matrix, a wafer, a pill, and the like. A depot may comprise a biopolymer. The biopolymer may be a sustained-release biopolymer. The depot may be deposited at or near, generally in close proximity, to a target site.

In an embodiment, the invention provides a method for treating a CNS disorder associated with a proinflammatory agent in a subject in need thereof. The method comprises administering to the subject an intracellular TNF modifying agent in an amount effective to treat the CNS disorder. The intracellular TNF modifying agent may be administered directly to the subject's CNS or may be administered peripherally, such as perispinally, intranasally, parentally, and the like. The method may further comprise administering an extracellular TNF modifying agent to enhance the treatment of the CNS disorder.

Various embodiments of the invention may provide one or more advantages. For example, as discussed herein, targeting the intracellular TNF cascade has several advantages over targeting soluble TNF and TNF receptors. The goal of blocking TNF and its downstream effector molecules in the brain through the use of intracellular modifying agents may provide greater efficacy, specificity, and avoid potentially deleterious effects of soluble TNF blocking agents in the brain. Furthermore, several intracellular TNF-modifying agents may be used in combination in order to direct the inhibition of TNF with more selectivity on the precise intracellular pathway-thereby avoiding apoptosis. These and other advantages will become evident to those of skill in the art upon reading the description provided herein.

### BRIEF DESCRIPTION OF THE DRAWINGS

FIG. 1 is a schematic diagram of TNF signal transduction.

FIG. 2 is a diagrammatic illustration of a patient's brain, the associated spaces containing cerebrospinal fluid, and the flow of cerebrospinal fluid in the subarachnoid space.

FIG. 3 is a diagrammatic illustration of a drug delivery system according to an embodiment of the present invention.

FIG. 4 is a diagrammatic illustration of a drug delivery system and a catheter implanted in a patient according to an embodiment of the present invention.

FIG. 5 is a diagrammatic illustration of a catheter implanted in a patient and a drug delivery system according to an embodiment of the present invention.

FIG. 6 is a diagrammatic illustration of a drug delivery system and catheter implanted in a patient according to an embodiment of the present invention.

FIG. 7 is a diagrammatic illustration of a drug delivery system comprising a sensor according to an embodiment of the present invention.

The figures are not necessarily to scale.

### DETAILED DESCRIPTION OF THE INVENTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

All scientific and technical terms used in this application have meanings commonly used in the art unless otherwise specified. The definitions provided herein are to facilitate understanding of certain terms used frequently herein and are not meant to limit the scope of the present disclosure.

In the context of the present invention, the terms “treat”, “therapy”, and the like mean alleviating, slowing the progression, preventing, attenuating, or curing the treated disease.

As used herein, “disease”, “disorder”, “condition” and the like, as they relate to a subject's health, are used interchangeably and have meanings ascribed to each and all of such terms.

As used herein, “subject” means a mammal undergoing treatment. Mammals include mice, rats, cats, guinea pigs, hampsters, dogs, horses, cows, monkeys, chimpanzees, and humans.

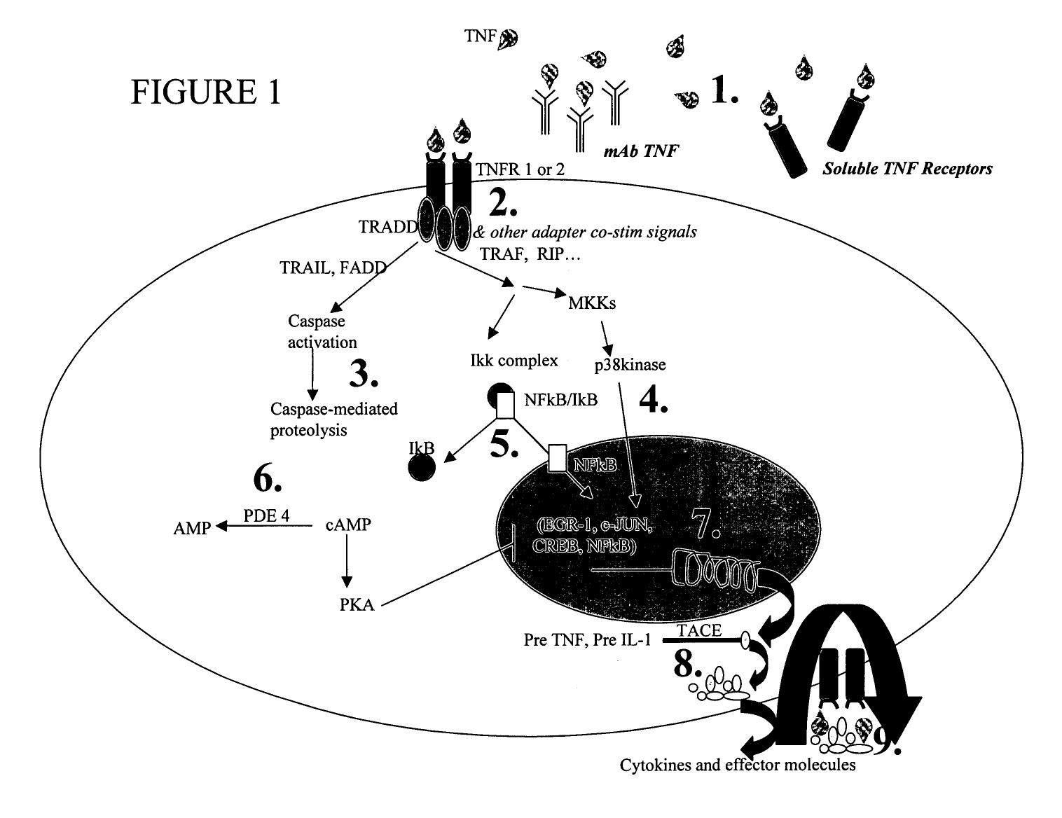

As used herein, “intracellular TNF modifying agent” means an agent that affects an intracellular molecule associated with signal transduction in the TNF inflammatory cascade and includes small molecule chemical agents and biological agents, such as polynucleotides and polypeptides, which include antibodies and fragments thereof, antisense, small interfering RNA (siRNA), and ribosymes. Nonlimiting examples of intracellular TNF modifying agents include agents that act at sites 2-8 shown in FIG. 1.

As used herein, “extracellular TNF modifying agent” means an agent that affects the action of TNF at a TNF cell surface receptor and agents that affect the action of secreted molecules associated with the TNF inflammatory cascade, such as IL-1, IL-6, and HMG-B1. Extracellular TNF modifying agents include small molecule chemical agents and biological agents, such as polynucleotides and polypeptides, which include antibodies and fragments thereof, antisense, small interfering RNA (siRNA), and ribosymes. Nonlimiting examples of intracellular TNF modifying agents include agents that act at sites 1 and 9 shown in FIG. 1.

As used herein, “TNF blocking agent” means any agent that has an inhibitory effect on TNF, its intracellular inflammatory cascade, and its associated secreted agents and includes intracellular and extracellular TNF modifying agents.

Delivery System

An embodiment of the invention provides a system for delivering a therapeutic composition comprising an intracellular TNF-signal transduction-modulating agent to a CNS of a subject in need thereof. The system comprises therapy delivery device and a catheter operably coupled to the therapy delivery device. The therapy delivery device may be a pump device. Non-limiting examples of pump devices include osmotic pumps, fixed-rate pumps, programmable pumps and the like. Each of the aforementioned pump systems comprise a reservoir for housing a fluid composition comprising a TNF blocking agent. The catheter comprises one or more delivery regions, through which the fluid may be delivered to one or more target regions of the subject. The pump device may be implantable or may be placed external to the subject.

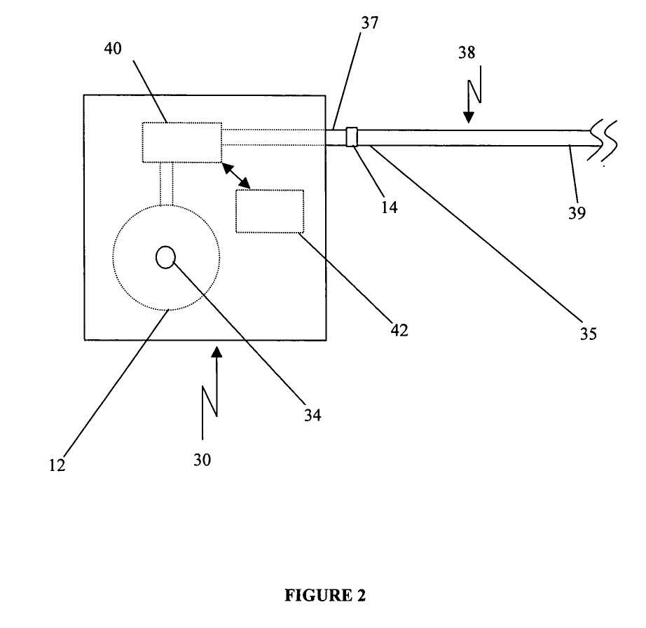

The therapy delivery device 30 shown in FIG. 2 comprises a reservoir 12 for housing a composition comprising a TNF blocking agent and a pump 40 operably coupled to the reservoir 12. The catheter 38 shown in FIG. 2 has a proximal end 35 coupled to the therapy delivery device 30 and a distal end 39 adapted to be implanted in a subject. Between the proximal end 35 and distal end 39 or at the distal end 39, the catheter 38 comprises one or more delivery regions (not shown) through which the TNF blocking agent may be delivered. The therapy delivery device 30 may have a port 34 into which a hypodermic needle can be inserted to inject a quantity of TNF blocking agent into reservoir 12. The therapy delivery device 30 may have a catheter port 37, to which the proximal end 35 of catheter 38 may be coupled. The catheter port 37 may be operably coupled to reservoir 12. A connector 14 may be used to couple the catheter 38 to the catheter port 37 of the therapy delivery device 30. The therapy delivery device 30 may be operated to discharge a predetermined dosage of the pumped fluid into a target region of a patient. The therapy delivery device 30 may contain a microprocessor 42 or similar device that can be programmed to control the amount of fluid delivery. The programming may be accomplished with an external programmer/control unit via telemetry. A controlled amount of fluid comprising a TNF blocking agent may be delivered over a specified time period. With the use of a programmable delivery device 30, different dosage regimens may be programmed for a particular patient. Additionally, different therapeutic dosages can be programmed for different combinations of fluid comprising therapeutics. Those skilled in the art will recognize that a programmed therapy delivery device 30 allows for starting conservatively with lower doses and adjusting to a more aggressive dosing scheme, if warranted, based on safety and efficacy factors.

If it is desirable to administer more than one therapeutic agent, such as one or more TNF blocking agent, the fluid composition within the reservoir 12 may contain a second, third, fourth, etc. therapeutic agent. Alternatively, the device 30 may have more than one reservoir 12 for housing additional compositions comprising a therapeutic agent. When the device 30 has more than one reservoir 12, the pump 40 may draw fluid from one or more reservoirs 12 and deliver the drawn fluid to the catheter 38. The device 30 may contain a valve operably coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the pump 40. A catheter 38 may have more than one lumen. Each lumen may be adapted to deliver a therapeutic agent from one or more reservoirs 12 of the device 30. It will also be understood that more than one device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference.

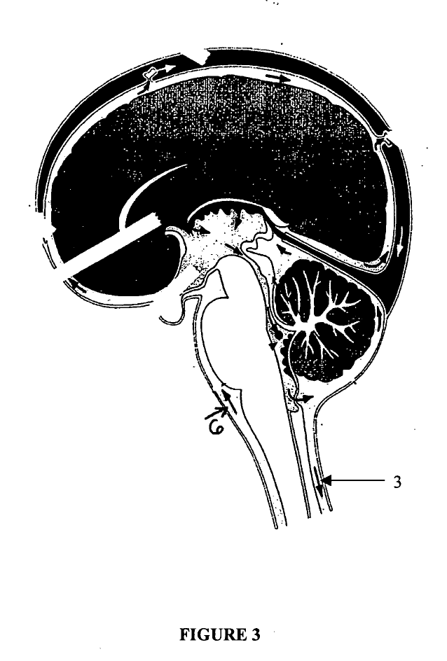

According to an embodiment of the invention, a composition comprising an intracellular TNF modifying agent may be delivered directly to cerebrospinal fluid 6 of a subject. Referring to FIG. 3, cerebrospinal fluid (CSF) 6 exits the foramen of Magendie and Luschka to flow around the brainstem and cerebellum. The arrows within the subarachnoid space 3 in FIG. 3 indicate cerebrospinal fluid 6 flow. The subarachnoid space 3 is a compartment within the central nervous system that contains cerebrospinal fluid 6. The cerebrospinal fluid 6 is produced in the ventricular system of the brain and communicates freely with the subarachnoid space 3 via the foramen of Magendie and Luschka. A composition comprising an intracellular TNF modifying agent may be delivered to cerebrospinal fluid 6 of a patient anywhere that the cerebrospinal fluid 6 is accessible. For example, the composition may be administered intrathecally or intracerebroventricularly.

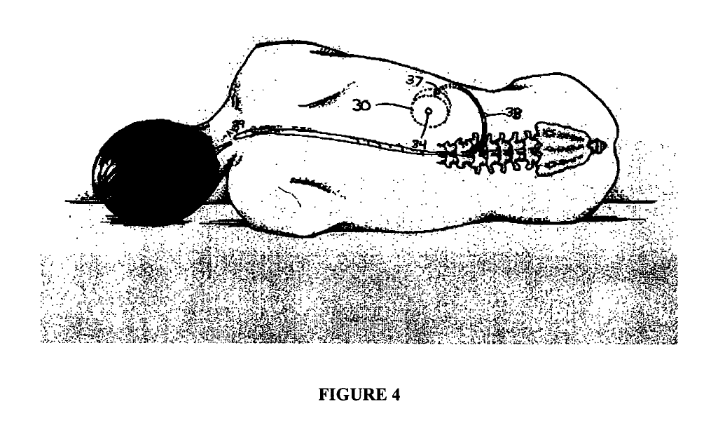

FIG. 4 illustrates a system adapted for intrathecal delivery of a composition comprising an intracellular TNF modifying agent. As shown in FIG. 4, a system or device 30 may be implanted below the skin of a patient. Preferably the device 30 is implanted in a location where the implantation interferes as little as practicable with patient activity. One suitable location for implanting the device 30 is subcutaneously in the lower abdomen. According to an embodiment of the invention, catheter 38 may be positioned so that the distal end 39 of catheter 38 is located in the subarachnoid space 3 of the spinal cord such that a delivery region (not shown) of catheter is also located within the subarachnoid space 3. It will be understood that the delivery region can be placed in a multitude of locations to direct delivery of a therapeutic agent to a multitude of locations within the cerebrospinal fluid 6 of the patient. The location of the distal end 39 and delivery region(s) of the catheter 38 may be adjusted to improve therapeutic efficacy. While device 30 is shown in FIG. 4, delivery of a composition comprising an intracellular TNF modifying agent into the CSF, for example for treating pain, can be accomplished by injecting the therapeutic agent via port 34 to catheter 38.

According to an embodiment of the invention, a composition comprising an intracellular TNF modifying agent may be delivered intraparenchymally directly to brain tissue of a subject. A therapy delivery device may be used to deliver the agent to the brain tissue. A catheter may be operably coupled to the therapy delivery device and a delivery region of the catheter may be placed in or near a target region of the brain.

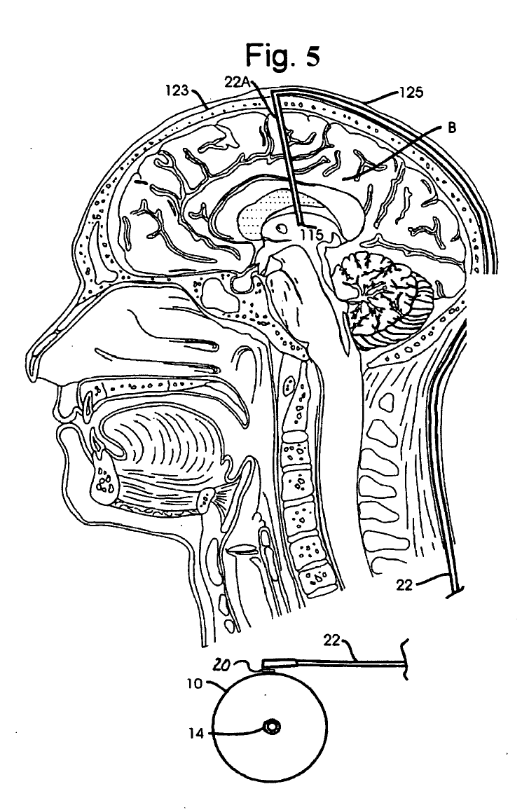

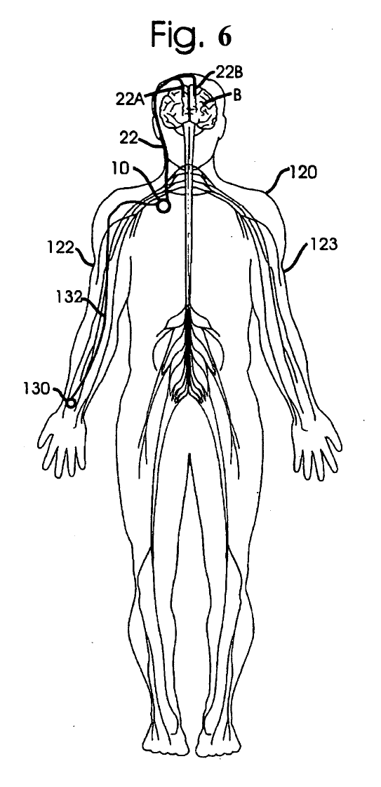

One suitable system for administering a therapeutic agent to the brain is discussed in U.S. Pat. No. 5,711,316 (Elsberry) as shown FIGS. 5 and 6 herein. Referring to FIG. 5, a system or therapy delivery device 10 may be implanted below the skin of a patient. The device 10 may have a port 14 into which a hypodermic needle can be inserted through the skin to inject a quantity of a composition comprising a therapeutic agent. The composition is delivered from device 10 through a catheter port 20 into a catheter 22. Catheter 22 is positioned to deliver the agent to specific infusion sites in a brain (B). Device 10 may take the form of the like-numbered device shown in U.S. Pat. No. 4,692,147 (Duggan), assigned to Medtronic, Inc., Minneapolis, Minn. The distal end of catheter 22 terminates in a cylindrical hollow tube 22A having a distal end 115 implanted into a target portion of the brain by conventional stereotactic surgical techniques. Additional details about end 115 may be obtained from pending U.S. application Ser. No. 08/430,960 entitled “Intraparenchymal Infusion Catheter System,” filed Apr. 28, 1995 in the name of Dennis Elsberry et at. and assigned to the same assignee as the present application. Tube 22A is surgically implanted through a hole in the skull 123 and catheter 22 is implanted between the skull and the scalp 125 as shown in FIG. 1. Catheter 22 is joined to implanted device 10 in the manner shown, and may be secured to the device 10 by, for example, screwing catheter 22 onto catheter port 20.

Referring to FIG. 6, a therapy delivery device 10 is implanted in a human body 120 in the location shown or may be implanted in any other suitable location. Body 120 includes arms 122 and 123. Catheter 22 may be divided into twin tubes 22A and 22B that are implanted into the brain bilaterally. Alternatively, tube 22B may be supplied with drugs from a separate catheter and pump.

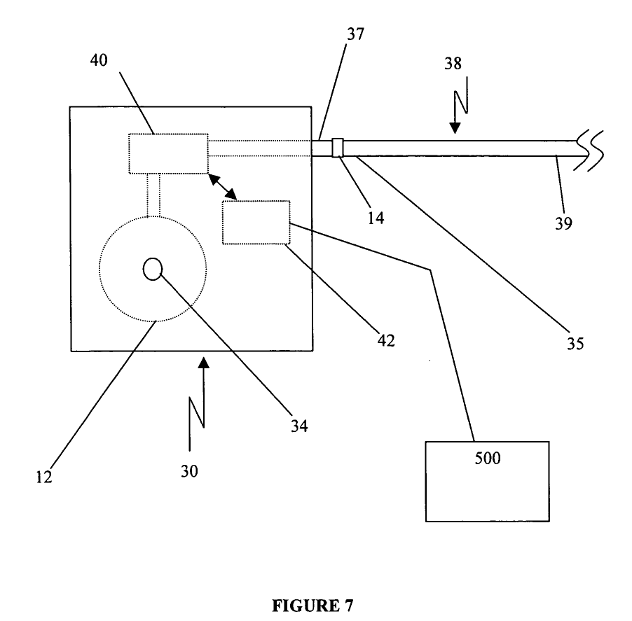

Referring to FIG. 7, therapy delivery device 30 may include a sensor 500. Sensor 500 may detect an event associated with a CNS disorder associated with an inflammatory immune response, such as a dysfunctional immune or sickness response, or treatment of the disorder, such as or whether an immune response has been attenuated or enhanced. Sensor 500 may relay information regarding the detected event, in the form of a sensor signal, to processor 42 of device 30. Sensor 500 may be operably coupled to processor 42 in any manner. For example, sensor 500 may be connected to processor via a direct electrical connection, such as through a wire or cable. Sensed information, whether processed or not, may be recoded by device 30 and stored in memory (not shown). The stored sensed memory may be relayed to an external programmer, where a physician may modify one or more parameter associated with the therapy based on the relayed information. Alternatively, based on the sensed information, processor 42 may adjust one or more parameters associated with therapy delivery. For example, processor 42 may adjust the amount and timing of the infusion of a TNF blocking agent. Any sensor 500 capable of detecting an event associated with an the disease to be treated or an inflammatory immune response may be used. Preferably, the sensor 500 is implantable. It will be understood that two or more sensors 500 may be employed.

Sensor 500 may detect a polypeptide associated with a CNS disorder or an inflammatory immune response; a physiological effect, such as a change in membrane potential; a clinical response, such as blood pressure; and the like. Any suitable sensor 500 may be used. In an embodiment, a biosensor is used to detect the presence of a polypeptide or other molecule in a patient. Any known or future developed biosensor may be used. The biosensor may have, e.g., an enzyme, an antibody, a receptor, or the like operably coupled to, e.g., a suitable physical transducer capable of converting the biological signal into an electrical signal. In some situations, receptors or enzymes that reversibly bind the molecule being detected may be preferred. In an embodiment, sensor 500 is capable of detecting an inflammatory cytokine. In an embodiment sensor 500 is capable of detecting TNF in cerebrospinal fluid. In an embodiment, sensor 500 may be a sensor as described in, e.g., U.S. Pat. No. 5,978,702, entitled TECHNIQUES OF TREATING EPILEPSY BY BRAIN STIMULATION AND DRUG INFUSION, which patent is hereby incorporated herein by reference in its entirety, or U.S. patent application Ser. No. 10/826,925, entitled COLLECTING SLEEP QUALITY INFORMATION VIA A MEDICAL DEVICE, filed Apr. 15, 2004, which patent application is hereby incorporated herein by reference in its entirety, or U.S. patent application Ser. No. 10/820,677, entitled DEVICE AND METHOD FOR ATTENUATING AN IMMUNE RESPONSE, filed Apr. 8, 2004.

In an embodiment, cerebrospinal levels of TNF are detected. A sample of CSF may be obtained and the levels of TNF in the sample may be detected by Enzyme-Linked Immunoabsorbant Assay (ELISA), microchip, conjugated fluorescence or the like.

Feedback to a therapy delivery device may be provided to alter infusion parameters of the TNF blocking agent.

TNF Blocking Agnets

An embodiment of the invention provides a method for treating a CNS disease or disorder associated with a pro-inflammatory agent by administering to the subject a composition comprising an intracellular TNF modifying agent. The discussion in the following numbered sections corresponds the same numbered portions of FIG. 1.

1. Extracellular TNF Modifying Agents.

While not an intracellular TNF-signal transduction modulating agent, an extracellular TNF modifying agent, such as a soluble TNF inhibitor, may be used in combination with an intracellular TNF-signal transduction modulating agent to treat a CNS disease or disorder. Examples of soluble TNF inhibitors include fusion proteins (such as etanercept); monoclonal antibodies (such as infliximab and D2E7); binding proteins (such as onercept); antibody fragments (such as CDP 870); CDP571 (a humanized monoclonal anti-TNF-alpha IgG4 antibody), soluble TNF receptor Type I, pegylated soluble TNF receptor Type I (PEGs TNF-R1) and dominant -negative TNF variants, such as DN-TNF and including those described by Steed et al. (2003), “Inactivation of TNF signaling by rationally designed dominant-negative TNF variants”, Science, 301 (5641): 1895-8. An extracellular TNF modifying agent may be administered to the subject either alone or in combination with an intracellular TNF-signal transduction-modulating agent.

With the signal transduction pathways becoming clearer, therapeutic agents that interfere with the specific intracellular actions of TNF may provide more specific therapeutic approaches to modulating TNF production. The remainder of the numbered sections below discuss attenuating TNF production and release through various intercellular approaches.

2. Inhibition of Related Cytoplasmic Proteins

The signals initiated by the TNF receptors are determined by the additional cytoplasmic proteins that are recruited to the TNF/TNFR complexes. The administration of agents that modulate the recruitment or binding of these cytoplasmic proteins can block the harmful effects of TNF while potentially allowing the beneficial effects to take place. There are several cytoplasmic proteins that propagate the signal leading to apoptosis or programmed cell death including death domain proteins, death effector domain proteins, TNF receptor-associated factors (TRAFs) and caspase recruitment domain proteins. For example, RDP58 (SangStat) is in clinical trials for Inflammatory Bowl Disease. RDP58 targets an important intra-cellular protein complex consisting of TRAFs. Next generation SangSat molecules aim to inhibit TNF synthesis and are being developed for IBD and other peripheral diseases. Other examples of agents that inhibit related signaling molecules include, but are not limited to, efalizumab (anti-LFA 1), antegren (natalizumab), CDP 232, CTLA-4Ig, rituximab I (anti-CD20 antibody), xanelim (anti-CD11b antibody).

Embodiment of the invention provide methods and devices to block the effects of TNF by administering agents that block the translocation or binding of death domain proteins, death effector domain proteins, TNF receptor-associated factors (TRAFs), and caspase recruitment domain proteins, to the TNF receptor complex. These agents may be administered to a targeted area or a targeted cell type to prevent the TNFa signal transduction cascade and thereby treat CNS disorders. The targeted delivery may be accomplished by using a drug delivery system comprising a therapy delivery device and an operably coupled catheter.

3. Anti-Apoptotic Agents

Extensive studies in post-mortem brain tissue of several neurodegenerative diseases revealed evidences of apoptotic cell death (Jellinger & Stadelmann, 2001, “Problems of cell death in neurodegeneration and Alzheimer's Disease”, J. Alzheimers Dis., 3(1):31-40) . The initiating signal for apoptosis is often TNF. TNF triggers downstream events that lead to glial cell activation and death, and nerve cell death, amounting to neurodegeneration. These events occur through the activation of caspases, key apoptosis-inducing enzymes important for the induction of cell death by TNFR ligation. Agents that prevent apoptotic events from taking place have shown efficacy in diseases of the periphery when administered peripherally. For greatest safety and efficacy in the brain, apoptosis inhibitors will require targeted delivery to the CNS. In an embodiment, the targeted delivery of caspase inhibitors using the drug delivery system is intraparenchymal.

Embodiments of the invention provide methods and devices to block the TNF-induced effects on apoptosis by administering agents that block apoptosis such as Pan-caspase inhibitor z-VAD, Pralnacasan (VX-740, Vertex), inhibitors of the inflammation target caspase-1(ICE), VX-765, VX-799, CV1013 (Maxim Pharmaceuticals), IDN 6556, IDN 6734 (Idun Pharmaceuticals-the first broad spectrum caspase inhibitor to be studied in humans), Activase, Retavase, TNKase (Metalyse, Tenecteplase, TNK-tPA), Pexelizumab, CAB2, RSR13 (Efaproxiral Sodium), VP025.

4. Kinase Inhibitors/Cell Signaling Inhibitors

Therapies that fall in this category are capable of manipulating the second messenger systems. Kinase activation signals multiple downstream effectors including those involving phosphatidylinositol 3-kinase and mitogen-activated protein kinases (MAPK), p38 MAPK, Src, and protein tyrosine kinase (PTK). Of particular importance in the signaling of TNFa effects is the downstream activation of MAPK. The majority of tyrosine kinase inhibitors have been developed to target solid tumors and cancer cells. For example, the tyrosine kinase inhibitors PTK787/ZK 222584 and GW572016 in clinical trials for malignant mesothelioma and metastatic breast cancer, respectively. Kinases such as Gleevec, Herceptin and Iressa are particularly popular targets in cancer therapy.

While the current route of administration for many of these agents is oral or parenteral, their effectiveness in the brain may require targeted delivery through a drug delivery system. Furthermore, intracellular targeted agents could be conjugated to cell specific marker to create a more localized and specific therapy.

Embodiments of the invention provide methods and devices to block the TNF-induced effects by administering a kinase inhibitor. An embodiment of this invention provides for the targeted delivery of a kinase inhibitor to a specific brain region with a drug delivery system. An example of a kinase inhibitor might be selected from Gleevec, Herceptin, Iressa, imatinib (ST1571), herbimycin A, tyrphostin 47, and erbstatin, genistein, staurosporine, PD98059, SB203580, CNI-1493, VX-50/702 (Vertex/Kissei), SB203580, BIRB 796 (Boehringer Ingelheim), Glaxo P38 MAP Kinase inhibitor, RWJ67657 (J&J), UO126,Gd, SCIO-469 (Scios), RO3201195 (Roche), Semipimod (Cyotkine PharmaSciences) or derivatives of the above mentioned agents. A conjugated molecule could consist of a cluster designator on an inflammatory cell or other receptor depending on the cell type determined to be the major contributor to enhanced TNF in a particular disease state. For example, substance P receptor for indications in pain.

WO2003072135A2 demonstrates that intracerebroventricular administration of CNI-1493 significantly inhibits LPS induced release of TNF. However to be therapeutically efficacious in neurodegenerative disorders it may require targeted intraparenchymal delivery through a drug delivery system.

Other kinase inhibitors whose mechanism of action has not been fully elucidated, but which inhibit inflammatory cascades may also be used according to the teachings of the present disclosure. One such kinase inhibitor is aminopyridazine (MW01-070C), which has been shown to suppress the production of IL-1b and INOS. See Watterson et al. (2002), “Discovery of new chemical classes of synthetic ligands that suppress neuroinflammatory responses”, Journal of Molecular Neuroscience; 19(1-2): 89-94.

5. NFKB Inhibition

NFκB is transcription factor involved in the production of cytokines and chemokines necessary for inflammation. Its complex but well described signaling function provides for several targeted therapeutic opportunities. As it turns out, several agents currently used to manage inflammatory conditions/diseases in the periphery indirectly diminish NFkB such as NSAIDS, asprin and corticosteroids. However, their lack of efficacy and their side effects have made it necessary to develop alternative ways and more direct routes of targeting NFκB. These direct approaches to target NFκB have not previously been suggested for use in neurological, neuropsychiatric or neurodegenerative disorders.

When inactive, NFκB is sequestered in the cytoplasm, bound by members of the IkappaB (IκB) family of inhibitor proteins. Once the appropriate signal is initiated (ie TNF binding to TNFR) IκB is degraded in a proteosome, leaving activated NFκB unsequestered. This causes the exposure of the nuclear localization signals (NLS) on the NFκB and the subsequent translocation of the molecule to the nucleus. Once in the nucleus, NFκB acts as a transcription factor, resulting in the transcription of several genes including TNFα and other pro-inflammatory factors. Agents that act to inhibit any of these steps involved in NFκB activation ultimately inhibit the destructive signal transduction cascade initiated by TNFα.

Embodiments of the invention provide methods and devices to block the TNF-induced effects by administering an IκB, and IKK or NFκb inhibitor. In an embodiment, the selection of an IκB, and IKK or NFκb inhibitor to be delivered to the brain using a drug delivery system is provided. An inhibtor may be selected from BMS345541 (IKK-B inhibitor, Bristol), Millennium NFκB of IKK-B inhibitor, pyrrolidine dithiocarbamatem (PDTC) derivatives, SPC600839 (Celgene/Serono), IKK-B inhibitor (Glaxo) and nuclear translocation inhibitors, such as deoxyspergualin (DSG).

6. PDE Inhibitors

Phosphodiesterase (PDE) inhibitors elevate Cyclic AMP (cAMP) levels by inhibiting its breakdown. Cyclic AMP regulates the release of TNFα by reducing the transcription of TNFα. Several phosphodiesterase inhibitors, particularly PDE IV inhibitors have been shown to reduce TNFαclinically when used to treat patients with asthma and COPD. However, their use in treating neurological, neurophsyciatric and neurodegenerative diseases has not been previously described. Additionally, their use in a targeted, delivery system, including a programmable drug delivery system, as described herein has not been previously described.

Embodiments of the invention provide methods and devices to block the TNF-induced effects by administering a PDE inhibitor. In an embodiment, the selection of a PDE IV inhibitor to be delivered to the brain using a therapy delivery system is provided. An inhibitor may be selected from Roflumilast, Arofylline, pentoxyfylline Ariflo (cilomilast, GSK), CDC-801 (Celgene), CD-7085 (Celgene), Rolipram, propenofylline.

7. Intranuclear Approaches

Gene silencing techniques (antisense, siRNA) and gene therapy approaches provide another means by which to inhibit or decrease the production of TNF. Gene silencing techniques may target the TNF gene directly or may target genes involved in apoptosis or other related signaling events as mentioned above (such as ISIS2302 and GI 129471). These agents may be used independently or in combination to modulate the expression of genes encoding TNF. Other intranuclear approaches such as crmA gene suppressive techniques may be applied.

TNF α antisense approaches are in clinical trials for the treatment of rheumatoid arthritis (Isis 104838), Crohn's disease (Isis 2302) for example. The method of targeted delivery of Isis 104838 or 2302 to a specific area of the brain has not been previously described.

In addition, the delivery of Isis 104838 or 2302 using a delivery system, such as a programmable therapy delivery system, as described herein has not been described.

WO 03/070897, “RNA Interference Mediated Inhibition Of TNF And TNF Receptor,” relates to compounds, compositions, and methods useful for modulating TNF associated with the development or maintenance of septic shock, rheumatoid arthritis, HIV and AIDS, psoriasis, inflammatory or autoimmune disorders, by RNA interference (RNAi), using short 1 5 interfering nucleic acid (siRNA) molecules. However, WO 03/070897 does not disclose the use of these techniques with a targeted administration route using a therapy delivery system.

An embodiment of the invention provides for the use of agents to block the transcription or translation of TNFa in neurological, neuropsychological and neurodegenerative conditions, brain injury or pain when administered with a targeted intraparenchymal drug delivery system and affecting the nucleus of brain cells.

8. TACE Inhibitors

TNFalpha converting enzyme (TACE) is the enzyme that generates the soluble form of TNF through a proteolytic cleavage event (26 kDa=>17 kDa). While both membrane-bound and soluble TNFα are biologically active, soluble TNFα is reported to be more potent. Agents that inhibit the intracellular TACE will ultimately decrease the amount of soluble TNF. Selective inhibitors of TACE are currently in clinical development to treat systemic inflammatory diseases such as arthritis through oral administration. However, the use of TACE inhibitors to treat neurological, neuropsychiatric, neurodegenerative disorders through targeted delivery to the brain using a drug delivery device has not been described.

In an embodiment, agents that inhibit TACE such as BMS561392 (Bristol-Myers Squibb), PKF242-484, PKF241-466 (Novartis), or other matrix metalloproteinase inhibitors are administered to treat neurological, neuropsychiatric and neurodegenerative diseases.

9. Inhibition of TNFα-Post Translational Effects

The initiation of TNFA signaling cascade results in the enhanced production of numerous factors that subsequently act in a paracrine and autocrine fashion to elicit further production of TNFα as well as other pro-inflammatory agents (IL-6, IL-1, HMG-B1). Extracellular TNF modifying agents that act on the signals downstream of TNF are being developed clinically for systemic inflammatory diseases. Some of these agents are designed to block other effector molecules while others block the cellular interaction needed to further induce their production (integrins, cell adhesion molecules etc). While their use outside of the brain to modulate TNFα-induced inflammatory cascade has been suggested previously, the administration of these agents to the brain with the use of targeted drug delivery systems to treat neurological, neuropsychiatric and neurodegenerative diseases has not been described.

An embodiment of the invention provides for the selection of an agent to inhibit the TNF-induced effects that are downstream of any TNF/TNFR complex effects. This agent is then delivered to the patient, to e.g. a specific brain region, using a drug delivery system to treat neurological, neuropsychiatric and neurodegenerative diseases. The agent may be selected from the following: integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, tocilizumab, Chugai), HMGB-1 mAb(Critical Therapeutics Inc.), anti-IL2R antibody (daclizumab, basilicimab), ABX (anti IL-8 antibody), recombinant human IL-10, HuMax IL-15 (anti-IL15 antibody).

Injectable Composition

The above-mentioned TNF blocking agents may be administered to a subject's CNS as injectable compositions. Injectable compositions include solutions, suspensions, dispersions, and the like. Injectable solutions or suspensions may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Solutions or suspensions comprising a therapeutic agent may be prepared in water, saline, isotonic saline, phosphate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is sterile, fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption--for example, aluminum monosterate hydrogels and gelatin. Excipients that increase solubility, such as cyclodextran, may be added.

Sterile injectable solutions may be prepared by incorporating a therapeutic agent in the required amount in the appropriate solvent with various other ingredients as enumerated above and, as required, followed by sterilization. Any means for sterilization may be used. For example, the solution may be autoclaved or filter sterilized. In the case of sterile powders for the preparation of sterile injectable solutions, the preferred methods of preparation are vacuum drying and freeze-drying techniques, which yield a powder of the active ingredient plus any additional desired ingredient present in a previously sterile-filtered solution.

Pharmaceutical Depot

In an embodiment, one or more of the above therapeutic agents may be placed in a pharmaceutical depot, such as a capsule, a microsphere, a particle, a gel, a coating, a matrix, a wafer, a pill, and the like. A depot may comprise a biopolymer. The biopolymer may be a sustained-release biopolymer. The depot may be deposited at or near, generally in close proximity, to a target site, such as a perispinal location. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

Dosage

Effective dosages for use in methods as described herein can be determined by those of skill in the art, particularly when effective systemic dosages are known for a particular therapeutic agent. Dosages may typically be decreased by at least 90% of the usual systemic dose if the therapeutic agent is provided in a targeted fashion. In other embodiments, the dosage is at least 75%, at least 80% or at least 85% of the usual system dose for a given condition and patient population. Dosage is usually calculated to deliver a minimum amount of one or more therapeutic agent per day, although daily administration is not required. If more than one pharmaceutical composition is administered, the interaction between the same is considered and the dosages calculated.

Intrathecal dosage, for example, can comprise approximately ten percent of the standard oral dosage. Alternatively, an intrathecal dosage is in the range of about 10% to about 25% of the standard oral dosage.

CNS Disorder

Embodiment of the invention provide methods and devices for treating a CNS disorder associated with a pro-inflammatory agent by administering to a subject a CNS disorder treating effective amount of a composition comprising an intracellular TNF modifying agent. CNS disorders associated with a pro-inflammatory agent include neurological, neurodegenerative, neuropsychiatric disorders, pain and brain injury. The intracellular TNF modifying agent may be administered directly to the CNS of the subject by, e.g., intrathecal (IT) delivery, intracerberalventricular (ICV) delivery, or intraparenchymal (IPA) delivery. Targeted delivery to the CNS avoids the potential for systemic immuno-suppression and other risk factors associated with systemic exposure to TNF blocking agents. In various embodiments, the intracellular TNF modifying agent is delivered to the CNS using a programmable pump, which allows for controlling the rate and time at which the agent is delivered and provides the ability to stop the delivery of the agent as desired. In various embodiments, an extracellular TNF modifying agent is also delivered to the subject to enhance the therapeutic effect of the intracellular TNF modifying agent.

Examples of various CNS disorders that may be treated and preferred delivery locations of therapeutic agents for treating the disorders is provided below.

1. Stroke

Blood-brain barrier breakdown and inflammation is observed in brain following stroke. Inflammatory processes are at least partly responsible for this breakdown. TNF blocking agents may be administered ICV, either chronically or transiently, following a stroke. In an embodiment, a TNF blocking agent is administered at the location of an infarct due to stroke. The location of the infarct may be identified by MRI or other know or future developed techniques. In an embodiment, the therapeutic agent is delivered to the middle cerebral artery at an infarct location or other cerebral artery distribution. Such delivery can be accomplished by placing a delivery region of a catheter in the artery and delivering the agent through the delivery region.

In addition to the ICV delivery of a TNF blocking agent at or near an infarct, a TNF blocking agent may be delivered IPA to an area surrounding the infarct to attenuate inflammation occurring in the ischemic periphery or penumbra that may lead to neurodegeneration if left untreated.

To attenuate the degeneration that occurs in a patient with hemiperesis following stroke a TNF blocking agent may be placed in the posterior limb of the internal capsule, for example.

In addition, a TNF blocking agent may be delivered to other brain regions that may be affected due to the secondary ischemic events following stroke, including but not limited to the pons, midbrain, medulla and the like.

Additional locations where a TNF blocking agent may be administered to treat stroke include locations where inflammatory events secondary to the initial stroke may occur.

For example middle cerebral artery stroke can produce a characteristic, cell-type specific injury in the striatum. Transient forebrain ischemia can lead to delayed death of the CAI neurons in the hippocampus. Therefore, a TNF blocking agent may be delivered to the striatum or hippocampus following a stroke event.

2. Alzheimer's Disease

Brain microvessels from Alzheimer's disease (AD) patients have been shown to express high levels of pro-inflammatory cytokines. It is suggested that inflammatory processes in the brain vasculature may contribute to plaque formation, neuronal cell death and neurodegeneration associated with AD. Accordingly, targeted delivery of a TNF blocking agent to a patient suffering from AD is contemplated herein.

In an embodiment, the TNF blocking agent is delivered in the vicinity of an amyloid plaque, where the inflammatory response in AD is mainly located. A TNF blocking agent may be administered IPA at the site of amyloid beta peptide accumulations, amyloid beta plaques, neurofibrillary tangles or other pathological sites associated with AD. For example, the affected area may be cortical or cerebellar and the plaques may be observed by imaging techniques known in the field.

Other IPA sites include the basal forebrain cholinergic system, a region that is vulnerable to degeneration in AD, the structures of the temporal lobe region, a region that is responsible for cognitive decline in AD patients, specifically the hippocampus, entorhinal cortex, and dentate gyrus.

3. Epilepsy

Blood-brain barrier breakdown and inflammation is observed in brain following seizures.

Inflammatory processes are at least partly responsible for this breakdown. In addition, TNF production is up-regulated during seizure-induced neuronal injury. In an embodiment TNF blocking agents are administered ICV, either chronically or transiently, following a seizure episode. In an embodiment, a TNF blocking agent is administered IPA to a seizure focus. In an embodiment, a TNF blocking agent is administered IPA to an area of the brain that undergoes neuronal injury, away from a specific seizure focus. For example, in patients with intractable temporal lobe epilepsy, the CAI region of the hippocampus undergoes pathophysiological changes associated with inflammatory processes and may ultimately result in neuronal cell loss in that region. Therefore, TNF blocking agents may be administered to the hippocampus in a epileptic patient. Other sites of IPA delivery are associated with brain regions affected by mesial temporal sclerosis such as the hippocampus or amygdala where evidence of inflammatory processes are often detected. Other structures in the CNS known to play a key role in the epileptogenic network such as the thalamus and subthalamic nucleus may also be targeted.

4. Depression

A TNF blocking agent may be administered ICV to target brain regions associated with inflammation in patients with depression. One suitable ICV location is the floor of the fourth ventricle, dorsal to the abducens nuclei, that contains serotonergic neurons.

In an embodiment, a TNF blocking agent is administered IPA to brain regions associated with the hypothalamic-pituitary-adrenal (HPA)-axis, as dysfunction of the HPA-axis is common in patients with depression. Furthermore, the cellular immune status in the brain regions associated with the HPA-axis is abnormal and is believed to be partly responsible for depressive symptoms. Elevations in proinflammatory cytokines such as TNF often found in depressed patients likely affect the normal functioning of the HPA axis. Examples of brain regions associated with the HPA-axis include, but are not limited to, the hypothalamus and the anterior pituitary gland.

In an embodiment, a TNF blocking agent is delivered to a brain region associated with serotonin production and output, since pro-inflammatory cytokines such as TNF may lower the circulating levels of serotonin-the mood stabilizing neurotransmitter. A TNF blocking agent delivered in a controlled fashion to the site of serotonin production may serve to regulate the production of serotonin thereby modulating the levels of serotonin production in patients with depression. The main site of serotonin production in the brain is the dorsal raphe nucleus. Other clusters or groups of cells that produce serotonin located along the midline of the brainstem may be targeted with IPA delivery of a TNF blocking agent. Main serotonergic nuclei may be targeted including the ventral surface of the pyramidal tract, the nucleus raphe obscurans, the raphe at the level of the hypoglossal nucleus, at the level of the facial nerve nucleus surrounding the pyramidal tract, the pontine raphe nucleus, above and between the longitudinal fasiculi at the central substantia grisea, the medial raphe nucleus, or the medial lemniscus nucleus.

5. Pain

A TNF blocking agent may be administered to a subject to treat pain in the subject. Any type of pain may be treated. In an embodiment, the pain is chronic pain. In various embodiments, the pain is chronic leg pain or chronic back pain. The TNF blocking agent may be administered intrathecally. In an embodiment, the TNF blocking agent is administered perispinally, which includes epidural, anatomic area adjacent the spine, intradiscal, subcutaneous, intramuscular, and intratendon administration. Generally, an agent administered perispinally to treat pain should be administered in close enough anatomic proximity to the pain fibers associated with the pain to reach the spine or subarachnoid space surrounding the pain fibers in the spinal cord in therapeutic concentration when administered perispinally. The TNF blocking agent may be administered perispinally in a pharmaceutical depot or via a delivery region of a catheter. The catheter may be operably coupled to a therapy delivery device. The optimal location of delivery of a TNF blocking agent for treating pain can readily be determined by one of skill in the art. Examples of locations for delivery for treatment of chronic back and leg pain can be found in, e.g., U.S. patent application Ser. No. 10/807,828, entitled INTRATHECAL GABAPENTIN FOR TREATMENT OF PAIN, filed Mar. 24, 2004.

All patents and publications referred to herein are hereby incorporated by reference in their entirety.

The teachings of the following patents and publications may be readily modified in light of the disclosure presented herein to produce the various devices described herein and to practice the various methods described herein:

Hirsch et al. (2003), “The role of glial reaction and inflammation in Parkinson's disease”,
Ann. N. Y. Acad. Sci.; 991: 214-28.
Ito, H. (2003), “Anti-interleukin-6 therapy for Crohn's disease”, Curr. Pharm. Des.; 9(4):
295-305.
WO 03/070897 RNA Interference Mediated Inhibition Of TNF And TNF Receptor
Superfamily Gene Expression Using Short Interfering Nucleic Acid (siNA)
U.S. Pat. No. 2003/0185826 Cytokine antagonists for the treatment of localized disorders
U.S. Pat. No. 6596747, Compounds derived from an amine nucleus and pharmaceutical
compositions comprising same
U.S. Pat. No. 6180355, Method for diagnosing and treating chronic pelvic pain syndrome
WO2003/072135, INHIBITION OF INFLAMMATORY CYTOKINE PRODUCTION
BY STIMULATION OF BRAIN MUSCARINIC RECEPTORS
WO98/20868, GUANYLHYDRAZONES USEFUL FOR TREATING DISEASES
ASSOCIATED WITH T CELL ACTIVATION
WO2002100330, METHODS OF ADMINISTERING ANTI-TNFα ANTIBODIES

1. A medical device comprising:
a pump;
a reservoir operably coupled to the pump;
an intracellular TNF modifying agent housed in the reservoir and being deliverable to a target site in a patient in an amount effective to treat a CNS disorder; and
a catheter operably coupled to the pump and configured to deliver the intracellular TNF modifying agent to the target site.

2. The medical device of claim 1, wherein the pump is a programmable pump.

3. The medical device of claim 1, wherein the pump is a fixed-rate pump.

4. The medical device of claim 1, wherein the pump is an osmotic pump.

5. The medical device of claim 1, wherein the intracellular TNF modifying agent is selected from the group consisting of an agent that blocks the translocation or binding of death domain proteins to the TNF receptor complex, an agent that blocks the translocation or binding of death effector domain proteins to the TNF receptor complex, and agent that blocks the translocation or binding of TNF receptor-associated factors (TRAFs) to the TNF receptor complex, an agent that blocks the translocation or binding of caspase recruitment domain proteins to the TNF receptor complex, an anti-apoptosis agent, a kinase inhibitor, a tyrosine kinase inhibitor, an NFκb inhibitor, an IκB inhibitor, an IKK inhibitor, a phosphodiesterase inhibitor, an agent that block the transcription or translation of TNFα, and a TACE inhibitor.

6. The medical device of claim 5, wherein the intracellular TNF modifying agent is selected from the group consisting of a SangStat molecule, RDP58, Efalizumab (anti-LFA 1), Antegren (natalizumab), CDP 232, CTLA-41g, Rituximab I (anti-CD20 antibody), Xanelim (anti-CD11b antibody), a caspase inhibitor, pan-caspase inhibitor z-VAD, Pralnacasan (VX-740, Vertex), an inhibitor of the inflammation target caspase-1(ICE), VX-765, VX-799, CV1013 (Maxim Pharmaceuticals), IDN 6556(Idun Pharmaceuticals), IDN 6734 (Idun Pharmaceuticals), Activase, Retavase, TNKase, Metalyse, Tenecteplase, TNK-tPA, Pexelizumab, CAB2, RSR13 (Efaproxiral Sodium), VP025, Gleevec, Herceptin, Iressa, Imatinib (ST1571), Herbimycin A, Tyrphostin 47, Erbstatin, Genistein, Staurosporine, PD98059, SB203580, CNI-1493, VX-50/702 (Vertex/Kissei), SB203580, BIRB 796 (Boehringer Ingelheim), Glaxo P38 MAP Kinase inhibitor, RWJ67657 (J&J), UO126, Gd, SCIO-469 (Scios), RO3201195 (Roche), Semipimod (Cyotkine PharmaSciences), BMS345541 (IKK-B inhibitor, Bristol), Millennium NFκB of IKK-B inhibitor, a pyrrolidine dithiocarbamatem (PDTC) derivative, SPC600839 (Celgene/Serono), an IKK-B inhibitor, a nuclear translocation inhibitors, deoxyspergualin (DSG), a PDE IV inhibitor, Roflumilast, Arofylline, Pentoxyfylline, Ariflo (cilomilast, GSK), CDC-801 (Celgene), CD-7085 (Celgene), Rolipram, Propenofylline, a TNF α antisense molecule, Isis 104838, Isis 2302, an siRNA targeted to TNF α mRNA, a matrix metalloproteinase inhibitor, BMS561392 (Bristol-Myers Squibb), PKF242-484 (Novartis), PKF241-466 (Novartis) and aminopyridazine (MW01-070C).

7. The medical device of claim 1, wherein the intracellular TNF modifying agent is selected from the group consisting of an agent that blocks the translocation or binding of death domain proteins to the TNF receptor complex, an agent that blocks the translocation or binding of death effector domain proteins to the TNF receptor complex, and agent that blocks the translocation or binding of TNF receptor-associated factors (TRAFs) to the TNF receptor complex, an agent that blocks the translocation or binding of caspase recruitment domain proteins to the TNF receptor complex, an anti-apoptosis agent, a kinase inhibitor, a tyrosine kinase inhibitor, an NFκb inhibitor, an IκB inhibitor, an IKK inhibitor, a phosphodiesterase inhibitor, an agent that block the transcription or translation of TNFα, and a TACE inhibitor.

8. The medical device of claim 7, wherein the intracellular TNF modifying agent is selected from the group consisting of a SangStat molecule, RDP58, Efalizumab (anti-LFA 1), Antegren (natalizumab), CDP 232, CTLA-41g, Rituximab I (anti-CD20 antibody), Xanelim (anti-CD11b antibody), a caspase inhibitor, pan-caspase inhibitor z-VAD, Pralnacasan (VX-740, Vertex), an inhibitor of the inflammation target caspase-1(ICE), VX-765, VX-799, CVI1013 (Maxim Pharmaceuticals), IDN 6556(Idun Pharmaceuticals), IDN 6734 (Idun Pharmaceuticals), Activase, Retavase, TNKase, Metalyse, Tenecteplase, TNK-tPA, Pexelizumab, CAB2, RSR13 (Efaproxiral Sodium), VP025, Gleevec, Herceptin, Iressa, Imatinib (ST1571), Herbimycin A, Tyrphostin 47, Erbstatin, Genistein, Staurosporine, PD98059, SB203580, CNI-1493, VX-50/702 (Vertex/Kissei), SB203580, BIRB 796 (Boehringer Ingelheim), Glaxo P38 MAP Kinase inhibitor, RWJ67657 (J&J), UO126, Gd, SCIO-469 (Scios), RO3201195 (Roche), Semipimod (Cyotkine PharmaSciences), BMS345541 (IKK-B inhibitor, Bristol), Millennium NFκB of IKK-B inhibitor, a pyrrolidine dithiocarbamatem (PDTC) derivative, SPC600839 (Celgene/Serono), an IKK-B inhibitor, a nuclear translocation inhibitors, deoxyspergualin (DSG), a PDE IV inhibitor, Roflumilast, Arofylline, Pentoxyfylline, Ariflo (cilomilast, GSK), CDC-801 (Celgene), CD-7085 (Celgene), Rolipram, Propenofylline, a TNF α antisense molecule, Isis 104838, Isis 2302, an siRNA targeted to TNF α mRNA, a matrix metalloproteinase inhibitor, BMS561392 (Bristol-Myers Squibb), PKF242-484 (Novartis), PKF241-466 (Novartis) and aminopyridazine (MW01-070C).

9. The medical device of claim 1, further comprising an extracellular TNF modifying agent deliverable via the catheter to the target site.

10. The medical device of claim 9, wherein the extracellular TNF modifying agent is housed in the reservoir.

11. The medical device of claim 9, further comprising a second reservoir, the second reservoir being operably coupled to the pump and housing the extracellular TNF modifying agent.

12. The medical device of claim 9, flurther comprising a second pump and a second reservoir, the second pump being operably coupled to the second reservoir, the catheter being operably coupled to the second pump and configured to deliver the extracellular TNF modifying agent to the target site.

13. The medical device of claim 1, further comprising a second catheter, a second pump and a second reservoir, the second reservoir being operably coupled to the second pump and housing an extracellular TNF modifying agent, the second catheter being operably coupled to the second pump and configured to deliver the extracellular TNF modifying agent to a second target site, wherein the target site within the patient and wherein the second target site are the same or different.

14. The medical device of claim 1, further comprising a sensor capable of detecting an event associated with the disorder or treatment of the disorder.

15. The medical device of claim 14, wherein the sensor is operably coupled to the pump.

16. The medical device of claim 15, wherein a parameter of the pump is capable of being modified by data from the sensor.

17. The medical device of claim 14, further comprising a memory operably coupled to the sensor and capable of storing sensed data.

18. The medical device of claim 14, wherein the sensor is capable of detecting a dysfunctional immune or sickness response or whether an inflammatory response has been attenuated or enhanced.

19. A method for treating a CNS disorder associated with a proinflammatory agent in a subject in need thereof, the method comprising:
administering an intracellular TNF modifying agent to the subject in an amount effective to treat the CNS disorder.

20. The method of claim 19, wherein administering the intracellular TNF modifying agent comprises administering the agent directly to the subject's central nervous system.

21. The method of claim 20, wherein administering the agent directly to the subject's central nervous system comprises administering the agent intraparenchymally.

22. The method of claim 20, wherein administering the agent directly to the subject's central nervous system comprises administering the agent intracerebroventricularly.

23. The method of claim 19, wherein administering the intracellular TNF modifying agent comprises peripherally administering the agent to the subject.

24. The method of claim 23, wherein peripherally administering the agent to the subject comprises perispinal, intranasal or parenteral administration

25. The method of claim 19, wherein the CNS disorder is a neurological disorder, a neurodegenerative disorder, a neuropsychiatric disorder, pain or brain injury.

26. The method of claim 19, wherein the CNS disorder is stroke.

27. The method of claim 26, wherein the administering an intracellular TNF modifying agent comprises administering the agent intrathecally.

28. The method of claim 26, wherein the administering an intracellular TNF modifying agent comprises administering the agent intracerebroventricularly.

29. The method of claim 26, wherein administering an intracellular TNF modifying agent comprises administering the agent to a cerebral artery of the subject.

30. The method of claim 29, wherein administering the agent to a cerebral artery comprises administering the agent to the middle cerebral artery.

31. The method of claim 30, wherein the agent administering the agent to the middle cerebral artery comprises administering the agent to the middle cerebral artery at or near infarct.

32. The method of claim 26, wherein the administering an intracellular TNF modifying agent comprises administering the agent intraparenchymally.

33. The method of claim 32, wherein administering the agent intraparenchymally comprises administering the agent at or near an infarct.

34. The method of claim 32, wherein administering the agent intraparenchymally comprises administering the agent at or near a site associated with secondary ischemic events following the stroke.

35. The method of claim 34, wherein administering the agent at or near a site associated with secondary ischemic events comprises administering the agent to the pons, midbrain, or medulla.

36. The method of claim 26, wherein the stroke is middle cerebral artery stroke.

37. The method of claim 36, wherein administering an intracellular TNF modifying agent comprises administering the agent intraparenchymally to the hippocampus.

38. The method of claim 37, wherein administering the agent intraparenchymally to the hippocampus comprises administering the agent intraparenchymally to the CA1 region of the hippocampus.

39. The method of claim 36, wherein administering an intracellular TNF modifying agent comprises administering the agent intraparenchymally to the striatum.

40. The method of claim 26, wherein the stroke results in hemiperesis.

41. The method of claim 40, wherein administering an intracellular TNF modifying agent comprises administering the agent intraparenchymally to the posterior limb of the internal capsule.

42. The method of claim 19, wherein the CNS disorder is Alzheimer's disease.

43. The method of claim 42, wherein the administering an intracellular TNF modifying agent comprises administering the agent intrathecally.

44. The method of claim 42, wherein the administering an intracellular TNF modifying agent comprises administering the agent intracerebroventricularly.

44. The method of claim 42, wherein the administering an intracellular TNF modifying agent comprises administering the agent intraparenchymally.

45. The method of claim 44, wherein the administering the agent intraparenchymally comprises administering the agent at or near an amyloid beta plaque.

46. The method of claim 44, wherein the administering the agent intraparenchymally comprises administering the agent to the basal forebrain cholinergic region.

47. The method of claim 44, wherein the administering the agent intraparenchymally comprises administering the agent to the temporal lobe region.

48. The method of claim 44, wherein the administering the agent intraparenchymally comprises administering the agent to the hippocampus.

49. The method of claim 44, wherein the administering the agent intraparenchymally comprises administering the agent to the entorhinal cortex

50. The method of claim 44, wherein the administering the agent intraparenchymally comprises administering the agent to the dentate gyrus.

51. The method of claim 19, wherein the CNS disorder is epilepsy.

52. The method of claim 51, wherein the administering an intracellular TNF modifying agent comprises administering the agent intrathecally.

53. The method of claim 51, wherein the administering an intracellular TNF modifying agent comprises administering the agent intracerebroventricularly.

54. The method of claim 51, wherein the administering an intracellular TNF modifying agent comprises administering the agent intraparenchymally.

55. The method of claim 54, wherein the administering the agent intraparenchymally comprises administering the agent at or near an epileptic focus.

56. The method of claim 54, wherein the administering the agent intraparenchymally comprises administering the agent to the hippocampus.

57. The method of claim 56, wherein administering the agent to the hippocampus comprises administering the agent to the CA1 region of the hippocampus.

58. The method of claim 19, wherein the CNS disorder is depression.

59. The method of claim 58, wherein the administering an intracellular TNF modifying agent comprises administering the agent intrathecally.

60. The method of claim 58, wherein the administering an intracellular TNF modifying agent comprises administering the agent intracerebroventricularly.

61. The method of claim 60, wherein the administering the agent intracerebroventricularly comprises administering the agent to the floor of the fourth ventricle, dorsal to the abducens nuclei.

62. The method of claim 58, wherein the administering an intracellular TNF modifying agent comprises administering the agent intraparenchymally.

63. The method of claim 62, wherein the administering the agent intraparenchymally comprises administering the agent to a brain region associated with the hypothalamic-pituitary-adrenal (HPA)-axis.

64. The method of claim 63, wherein administering the agent to a brain region associated with the HPA-axis comprises administering the agent to the hypothalamus.

65. The method of claim 63, wherein administering the agent to a brain region associated with the HPA-axis comprises administering the agent to the anterior pituitary gland.

66. The method of claim 62, wherein the administering the agent intraparenchymally comprises administering the agent to a brain region associated with serotonin production or output.

67. The method of claim 66, wherein administering the agent to a brain region associated with serotonin production or output comprises administering the agent to the dorsal raphe nucleus.

68. The method of claim 66, wherein administering the agent to a brain region associated with serotonin production or output comprises administering the agent to the midline of the brainstem.

69. The method of claim 66, wherein administering the agent to a brain region associated with serotonin production or output comprises administering the agent to a brain region selected from the group consisting of the ventral surface of the pyramidal tract, the nucleus raphe obscurans, the raphe at the level of the hypoglossal nucleus, at the level of the facial nerve nucleus surrounding the pyramidal tract, the pontine raphe nucleus, above and between the longitudinal fasiculi at the central substantia grisea, the medial raphe nucleus, and the medial lemniscus nucleus.

70. The method of claim 19, further comprising administering an extracellular TNF modifying agent to the subject in an amount effective to enhance treatment of the CNS disorder.

71. The method of claim 70, wherein administering an extracellular TNF modifying agent to the subject comprises administering an agent selected from the group consisting of TNF fusion protein, an antibody directed to TNF, a monoclonal antibody directed to TNF, a TNF binding protein, a soluble TNF receptor, a soluble pegylated TNF receptor, an antibody fragment directed to TNF, a dominant-negative TNF variant, an integrin antagonists, alpha-4 beta-7 integrin antagonists, a cell adhesion inhibitor, interferon gamma antagonists, a CTLA4-Ig agonists/antagonists, a CD40 ligand antagonists, a anti-IL-6 antibody, an anti-HMGB-1 antibody, an anti-IL2R antibody, an anti-IL-8 antibody, and an anti-IL-10 antibody.

72. The method of claim 70, wherein administering an extracellular TNF modifying agent to the subject comprises administering an agent selected from the group consisting of etanercept, infliximab, D2E7, onercept, CDP 870, CDP 571, PEGs TNF-R1, DN-TNF, BMS-188667, tocilizumab (Chugai), daclizumab, basilicimab, ABX (anti IL-8 antibody), and HuMax IL-15 (anti-IL15 antibody).

73. The method of claim 72, wherein administering an extracellular TNF modifying agent to the subject comprises administering the agent via a controlled administration system.

74. The method of claim 73, wherein the administering the agent via a controlled administration system comprises administering the agent in a depot.

75. The method of claim 74, wherein the administering the agent via a controlled administration system comprises administering the agent via a catheter operably coupled to a pump.

76. The method of claim 19, wherein the CNS disorder is pain.

77. The method of claim 76, wherein the pain is chronic pain.

78. The method of claim 76, wherein administering an intracellular TNF modifying agent comprises administering the agent intrathecally.

79. The method of claim 76, wherein administering an intracellular TNF modifying agent comprises administering the agent perispinally.

80. The method of claim 79, wherein administering the agent perispinally comprises administering the agent into a verterbral disc.

81. A pharmaceutical depot comprising:
a pharmaceutical delivery formulation;
an intracellular TNF modifying agent disposed in or on the pharmaceutical delivery formulation; and
an extracellular TNF modifying agent disposed in or on the pharmaceutical delivery formulation.

82. The depot of claim 81, wherein the pharmaceutical delivery formulation comprises a capsule, a microsphere, a particle, a gel, a coating, a matrix, a wafer, or a pill.

83. The depot of claim 82, wherein the pharmaceutical delivery formulation comprises a biopolymer.

84. The depot of claim 83, wherein the pharmaceutical polymer comprises a polymer selected from the group consisting of poly(alpha-hydroxy acid), poly(lactide-co-glycolide) (PLGA), polylactide (PLA), polyglycolide (PG), polyethylene glycol (PEG) conjugates of a poly(alpha-hydroxy acid), polyorthoester, polyaspirin, polyphosphagene, collagen, starch, chitosan, gelatin, alginate, dextran, vinylpyrrolidone, polyvinyl alcohol (PVA), PVA-g-PLGA, PEGT-PBT copolymer (polyactive), methacrylate, poly(N-isopropylacrylamide), PEO-PPO-PEO (pluronics), PEO-PPO-PAA copolymer, and PLGA-PEO-PLGA.

85. The depot of claim 81, wherein the intracellular TNF modifying agent is selected from the group consisting of an agent that blocks the translocation or binding of death domain proteins to the TNF receptor complex, an agent that blocks the translocation or binding of death effector domain proteins to the TNF receptor complex, and agent that blocks the translocation or binding of TNF receptor-associated factors (TRAFs) to the TNF receptor complex, an agent that blocks the translocation or binding of caspase recruitment domain proteins to the TNF receptor complex, an anti-apoptosis agent, a kinase inhibitor, a tyrosine kinase inhibitor, an NFκb inhibitor, an IκB inhibitor, an IKK inhibitor, a phosphodiesterase inhibitor, an agent that block the transcription or translation of TNFα, and a TACE inhibitor.

86. The depot of claim 81, wherein the intracellular TNF modifying agent is selected from the group consisting of a SangStat molecule, RDP58, Efalizumab (anti-LFA 1), Antegren (natalizumab), CDP 232, CTLA-41g, Rituximab I (anti-CD20 antibody), Xanelim (anti-CD11b antibody), a caspase inhibitor, pan-caspase inhibitor z-VAD, Pralnacasan (VX-740, Vertex), an inhibitor of the inflammation target caspase-1(ICE), VX-765, VX-799, CV1013 (Maxim Pharmaceuticals), IDN 6556(Idun Pharmaceuticals), IDN 6734 (Idun Pharmaceuticals), Activase, Retavase, TNKase, Metalyse, Tenecteplase, TNK-tPA, Pexelizumab, CAB2, RSR13 (Efaproxiral Sodium), VP025, Gleevec, Herceptin, Iressa, Imatinib (ST1571), Herbimycin A, Tyrphostin47, Erbstatin, Genistein, Staurosporine, PD98059, SB203580, CNI-1493, VX-50/702 (Vertex/Kissei), SB203580, BIRB 796 (Boehringer Ingelheim), Glaxo P38 MAP Kinase inhibitor, RWJ67657 (J&J), UO126, Gd, SCIO-469 (Scios), RO3201195 (Roche), Semipimod (Cyotkine PharmaSciences), BMS345541 (IKK-B inhibitor, Bristol), Millennium NFκB of IKK-B inhibitor, a pyrrolidine dithiocarbamatem (PDTC) derivative, SPC600839 (Celgene/Serono), an IKK-B inhibitor, a nuclear translocation inhibitors, deoxyspergualin (DSG), a PDE IV inhibitor, Roflumilast, Arofylline, Pentoxyfylline, Ariflo (cilomilast, GSK), CDC-801 (Celgene), CD-7085 (Celgene), Rolipram, Propenofylline, a TNF α antisense molecule, Isis 104838, Isis 2302, an siRNA targeted to TNF α mRNA, a matrix metalloproteinase inhibitor, BMS561392 (Bristol-Myers Squibb), PKF242-484 (Novartis), PKF241-466 (Novartis) and aminopyridazine (MW01-070C).

87. The method of claim 81, wherein administering an extracellular TNF modifying agent to the subject comprises administering an agent selected from the group consisting of TNF fusion protein, an antibody directed to TNF, a monoclonal antibody directed to TNF, a TNF binding protein, a soluble TNF receptor, a soluble pegylated TNF receptor, an antibody fragment directed to TNF, a dominant-negative TNF variant, an integrin antagonists, alpha-4 beta-7 integrin antagonists, a cell adhesion inhibitor, interferon gamma antagonists, a CTLA4-Ig agonists/antagonists, a CD40 ligand antagonists, a anti-IL-6 antibody, an anti-HIMGB-1 antibody, an anti-IL2R antibody, an anti-IL-8 antibody, and an anti-IL-10 antibody.

88. The method of claim 8 1, wherein administering an extracellular TNF modifying agent to the subject comprises administering an agent selected from the group consisting of etanercept, infliximab, D2E7, onercept, CDP 870, CDP 571, PEGs TNF-R1, DN-TNF, BMS-188667, tocilizumab (Chugai), daclizumab, basilicimab, ABX (anti IL-8 antibody), and HuMax IL-15 (anti-IL15 antibody).
